(19) United States
(12) Patent Application Publication
(10) Pub. No.: US 2016/0362632 A1
(43) Pub. Date: Dec. 15, 2016

(54) FLAVOLIPIDS AS SURFACTANTS IN CLEANSING COMPOSITIONS

(71) Applicants: Henkel AG & Co. KGaA, Duesseldorf (DE); The Dial Corporation, Scottsdale, AZ (US)

(72) Inventors: ALEXANDER SCHULZ, DUESSELDORF (DE); MICHAEL DREJA, NEUSS (DE); PETER SCHMIEDEL, DUESSELDORF (DE); MENGTAO PETE HE, SCOTTSDALE, AZ (US)

(21) Appl. No.: 14/740,059

(22) Filed: Jun. 15, 2015

(51) Int. Cl. C11D 1/66; C11D 3/386; A61Q 5/02; A61K 8/44; A61Q 19/10

(52) U.S. Cl. CPC C11D 1/66; A61K 8/442; A61Q 19/10; A61Q 5/02; C11D 3/38645; C11D 3/386; A61K 2800/75

(57) ABSTRACT

Cosmetic, detergent, and surface cleansing compositions are provided. The provided compositions include a flavolipid according to formula (I),

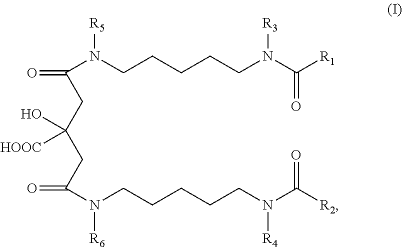

in which R₁ and R₂, independently of one another, are selected from linear or branched C₄-C₂₀ alkyl radicals, linear or branched C₄-C₂₀ alkenyl radicals, and linear or branched C₄-C₂₀ hydroxyalkyl radicals; R₃, R₄, R₅ and R₆, independently of one another, are selected from hydrogen, hydroxyl, C₁-C₄ alkyl radicals, C₁-C₄ alkenyl radicals, C₁-C₄ alkoxy radicals, and C₁-C₄ hydroxyalkyl radicals.

### FIELD OF THE INVENTION

The present invention generally relates to biosurfactants, and more particularly relates to cleansing compositions containing flavolipids as biosurfactants.

### BACKGROUND OF THE INVENTION

Cleansing compositions can take a variety of forms. For example, cleansing compositions include skin cleansers, hair cleansers, hair conditioners, laundry detergent, dishwashing detergent, household cleaning solutions, and a myriad of other such compositions. The ability of a cleansing composition to perform its function may be determined by the surfactant(s) that are included in the composition. Each type of surfactant has a range of chemical properties that makes that type of surfactant particularly suitable for certain applications, and less suitable for other applications. A combination of surfactants may be able to provide advantages to the cleansing composition.

Ecological sustainability has also become an important factor in cleansing compositions. Ecological sustainability operates both in regards to the production of the cleansing compositions and the ecological consequences of waste products containing the cleansing compositions. The inclusion of biosurfactants in cleansing compositions may benefit the ecological sustainability of cleansing compositions, both in regards to production and ecological effects of waste products.

Specifically, in regards to production, biosurfactants may be partially or wholly produced by biological systems. Biological sources for biosurfactants include plants, animals and microorganisms. Biosurfactants produced by microorganisms may have certain advantages, in part because sustainability can be readily achieved by selecting a sustainable feedstock for the microorganisms.

Biosurfactants may also minimize the ecological effects of waste products. Because biosurfactants are partially or wholly biologically produced, the ecological impact of biosurfactants in waste products may be less than that of other surfactants. Additionally, biosurfactants may be more susceptible to biodegradation, further decreasing the ecological effects of waste products.

Certain stains, such as those which include an inorganic component, may be especially difficult to remove. Examples of such stains include stains that contain iron or aluminum cations, such as stains from berries or anti-perspirant. Inorganic components are often difficult to dissolve, which may contribute to such stains being difficult to remove. Some biosurfactants may be able to improve the removal of such stains, which may operate by removing the metallic cation.

Certain surfactants may contribute to the drying or irritation of skin, which may result from the removal of the natural oils on skin which provide a protective layer. Some surfactant-containing compositions have attempted to address the removal of natural oils by including humectants, conditioning agents, and/or emollients to replace the natural oils, but the efficacy of these approaches varies based on an individual's skin. However, the use of biosurfactants may also reduce the drying or irritation effect of a surfactant-containing composition.

Accordingly, it is desirable to provide a surfactant composition that includes a number of biosurfactants, in order to more effectively combat difficult stains and/or drying/irritation of skin. In addition, it is desirable to provide biosurfactants that are generated sustainably, and which have a minimal ecological impact. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A surfactant composition is provided. The composition includes a number of flavolipids according to formula (I), or physiologically acceptable salts thereof,

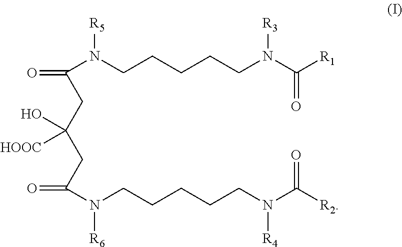

The number of flavolipids of formula (I) are selected such that R₁ and R₂, independently of one another, are selected from linear or branched C₄-C₂₀ alkyl radicals, linear or branched C₄-C₂₀ alkenyl radicals, and linear or branched C₄-C₂₀ hydroxyalkyl radicals; R₃, R₄, R₅ and R₆, independently of one another, are selected from hydrogen, hydroxyl, C₁-C₄ alkyl radicals, C₁-C₄ alkenyl radicals, C₁-C₄ alkoxy radicals, and C₁-C₄ hydroxyalkyl radicals. The composition also includes a number of additional surfactants and a carrier selected from a carrier fluid and a builder.

A cleansing composition is provided for cleansing textiles or surfaces. The composition includes a number of flavolipids according to formula (I), or salts thereof,

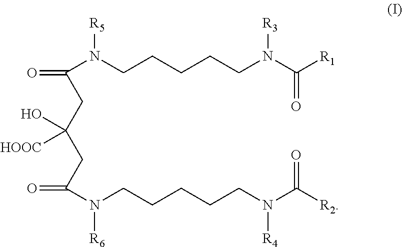

The number of flavolipids of formula (I) are selected such that R₁ and R₂, independently of one another, are selected from linear or branched C₄-C₂₀ alkyl radicals, linear or branched C₄-C₂₀ alkenyl radicals, and linear or branched C₄-C₂₀ hydroxyalkyl radicals; R₃, R₄, R₅ and R₆, independently of one another, are selected from hydrogen, hydroxyl, C₁-C₄ alkyl radicals, C₁-C₄ alkenyl radicals, C₁-C₄ alkoxy radicals, and C₁-C₄ hydroxyalkyl radicals. The composition also includes a liquid carrier.

A consumer product is provided for use as a laundry detergent. The consumer product includes a number of flavolipids according to formula (I), or salts thereof,

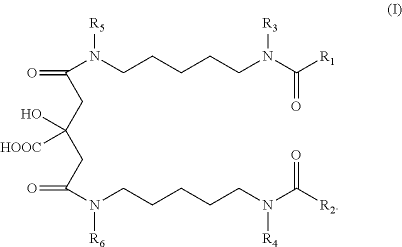

The number of flavolipids of formula (I) are selected such that R₁ and R₂, independently of one another, are selected from linear or branched C₄-C₂₀ alkyl radicals, linear or branched C₄-C₂₀ alkenyl radicals, and linear or branched C₄-C₂₀ hydroxyalkyl radicals; R₃, R₄, R₅ and R₆, independently of one another, are selected from hydrogen and hydroxyl. The composition also includes a number of anionic surfactants, a number of nonionic surfactants, a number of enzymes selected from proteases, amylases and cellulases, a number of builders and a liquid carrier.

### BRIEF DESCRIPTION OF THE DRAWINGS

The present invention will hereinafter be described in conjunction with the following drawing figures, wherein like numerals denote like elements, and

FIG. 1 is a diagram of an exemplary container dispensing a cosmetic composition that includes a flavolipid, according to an example of the principles described herein;

FIG. 2 is a diagram of an exemplary container holding a detergent composition that includes a flavolipid, according to an example of the principles described herein; and

FIG. 3 is a diagram of an exemplary container dispensing a surface cleansing composition that includes a flavolipid, according to an example of the principles described herein.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Ecological sustainability has become an important factor in the production of consumer products. Cleansing agents, in particular, have experienced increased pressure to employ “green” chemistry, due to the volume of cleansing agents that are used, as well as that the cleansing agents may be released into the environment following use. Surfactants may be one of the defining elements to cleansing agents, as surfactants may be the source of the cleansing power of the agent. Surfactants act to solubilize uncharged molecules, such as oils, in order to clean a surface, a textile, hair, skin, and the like.

Considerable attention has been given to the use of surfactants that minimize the detrimental effects to the environment upon release; however, less attention has been given to the production of surfactants in ways that are ecologically sustainable. The present specification is directed to providing cleansing compositions that provide improved ecological sustainability both during production and following use and release. The efficacy of a cleansing solution is also significant to consumer acceptance, such cleansing compositions should therefore also be able to provide effective and efficient cleaning.

A surfactant may have a hydrophobic end and a hydrophilic end. The hydrophobic end may allow the surfactant to interact with uncharged molecules, such as oils. The hydrophilic end may facilitate the interaction of the molecule with charged or polar molecules, such as water. The hydrophilic end may be used to classify surfactants, which may be anionic, cationic, nonionic, amphoteric, or zwitterionic. Anionic surfactants may have a negatively charged hydrophilic end. Examples of anionic surfactants include sulfate, sulfonate, carboxylate, phosphate, or the like. Anionic surfactants may be sensitive to water hardness. Cationic surfactants may be those that have a positively charged hydrophilic end, such as a quaternary amine. Nonionic surfactants may have a hydrophilic end which may be charge neutral, such as an ethoxylate, glycoside, or poly-ol; such surfactants may not be sensitive to water hardness. Amphoteric surfactants may be those that have a hydrophilic end which has a functional group that is capable of acting as a base, and a functional group that is capable of acting as an acid, such as amine oxides. Zwitterionic surfactants may have both a positive and negative charge on their hydrophilic ends, such as sultaines, or betaines. The hydrophobic end may include a saturated or unsaturated, linear or branched, substituted or unsubstituted, cyclic or acyclic alkyl chain containing at least 8 carbon atoms, which may also bear a number of heteroatoms.

In addition to the classification of surfactants by the charge of the hydrophilic end, surfactants may also be classified by the source from which they are isolated. A number of surfactants are prepared by a sequence of chemical reactions. In some cases, this sequence can be simple, such as the hydrolysis of a triglyceride (saponification) to release glycerol and fatty acids. In other cases, the preparation of surfactants may be more complex, such as the preparation of alkylbenzenesulfonates from tetramerized propylene and benzene with subsequent sulfonation. Another type of surfactants are biosurfactants, which are prepared from biological materials. Biosurfactants may be isolated from their natural source, and in some cases may be chemically treated to adjust the surfactant properties. It may also be possible to make biosurfactants synthetically, by a sequence of chemical reactions. The isolation of biosurfactants from natural sources may be able to provide an ecologically sustainable source of surfactants to be used in a variety of materials.

There are a variety of biosurfactants. Biosurfactants produced by microorganisms including semi-synthetic agents are of particular interest, due to the ability to produce large amounts of material in relatively short times, as well as the ability to employ sustainable feedstocks. Such biosurfactants include glycolipids, such as rhamnolipids and sophorolipids; lipopeptides, such as surfactin and lichenysin; and polymeric surfactants, such as emulsan and liposan.

Flavolipids are a type of biosurfactant. Flavolipids are a class of biosurfactants, some of which are produced by Flavobacterium sp. For the purposes of the present specification, a flavolipid may be a compound according to formula (I),

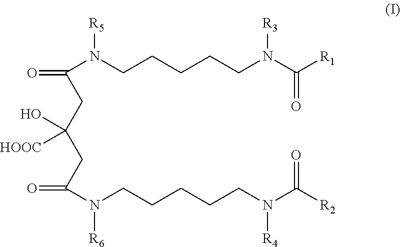

in which:

R₁ and R₂, independently of one another, are selected from: linear or branched C₄-C₂₀ alkyl radicals, linear or branched C₄-C₂₀ alkenyl radicals, and linear or branched C₄-C₂₀ hydroxyalkyl radicals;

R₃, R₄, R₅ and R₆, independently of one another, are selected from hydrogen, hydroxyl, C₁-C₄ alkyl radicals, C₁-C₄ alkenyl radicals, C₁-C₄ alkoxy radicals, and C₁-C₄ hydroxyalkyl radicals.

For the purposes of the present specification, flavolipids include compounds of formula (I) that are naturally produced, synthetically produced, and semisynthetically produced. Flavolipids of formula (I) may be able to act as chelating agents, in addition to their role as surfactants. This dual action of flavolipids according to formula (I) may provide a cleansing composition with increased efficacy at a lower production cost, as well as employing a higher quantity of ecologically sustainable materials.

Naturally produced flavolipids may be flavolipids which are produced naturally by a microorganism, such as Flavobacterium sp. Naturally produced flavolipids may be compounds according to formula (I), and include compounds in which R₃ and R₄ are both hydroxyl groups, and R₅ and R₆ are hydrogen. Naturally produced flavolipids may present a variety of R₁ and R₂ groups; it is possible to use both pure compounds and heterogenous mixtures, especially in such cases where separation to homogeneity may be difficult due to the presence of multiple similar R₁ and R₂ groups in the ensemble. Examples of R₁ and R₂ groups which may be present in a naturally produced flavolipid include the groups shown in formulae (IIa)-(IIe) and (IIIa)-(IIIe), below.

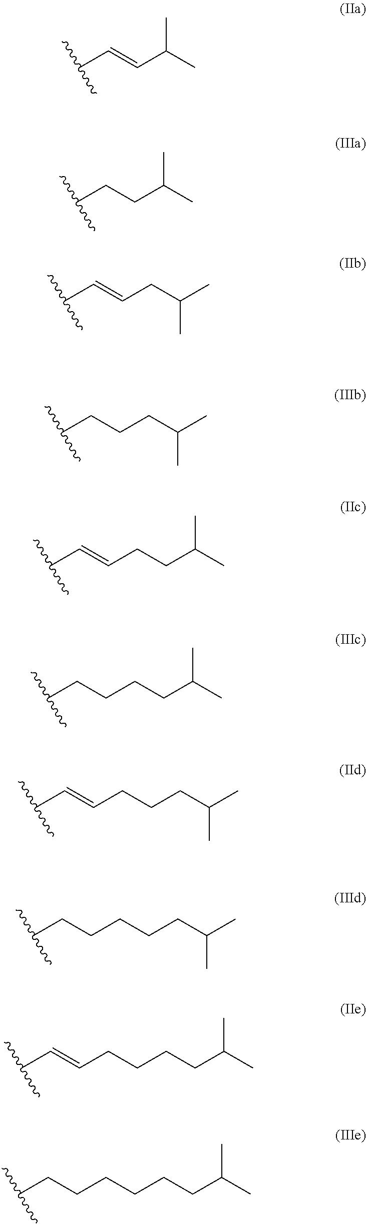

Flavolipids according to formula (I) may also be produced synthetically. Such synthetic production may be accomplished by a number of methods. In one example, flavolipids may be synthesized by sequential reactions between an acid halide, a cadaverine derivative (such as N-protected 5-hydroxyamino-pentan-1-amine derivatives), and citric acid; differential or orthogonal protection of functional groups may be used during the synthesis to facilitate the localization of the hydroxamic acid functionality.

Synthetic production of flavolipids may allow access to a great variety of flavolipids, including a variety of R₁ and R₂ groups. The choice of a variety of R₁ and R₂ groups may allow for the modulation of a number of surfactant properties, such as lipophilicity, ability to remove certain types of oils or stains, and the like. Synthetic production of flavolipids may also allow for tailoring of the chelating or foaming properties of the flavolipid by adjusting the R₃, R₄, R₅, and R₆ groups.

For example, a synthetically produced flavolipid could include a compound according to formula (I) in which R₃ and R₄ are hydrogen, R₅ and R₆ are hydroxyl, and R₁ and R₂ are chosen from formulae (IIa)-(IIe). According to another example, a synthetically produced flavolipid could include a compound according to formula (I) in which R₃ and R₅ are hydroxyl, R₄ and R₆ are hydrogen, and R₁ and R₂ are chosen from caprylyl, decyl, lauryl and myristyl groups. According to a further example, a synthetically produced flavolipid could include a compound according to formula (I) in which R₃ and R₄ are hydrogen, R₅ and R₆ are hydroxyl, and R₁ and R₂ are chosen from cetyl, stearyl, arachidyl and behenyl groups. In a still further example, a synthetically produced flavolipid could include a compound according to formula (I) in which R₃ and R₆ are hydrogen, R₄ and R₅ are hydroxyl, and R₁ and R₂ are chosen from isocaprylyl, isodecyl, isolauryl, isomyristyl, isocetyl, isostearyl, isoarachidyl, and isobehenyl groups. In another example, R₁ and R₂ may be chosen from isohexyl, isoheptyl, isocaprylyl and isodecyl groups, R₃ and R₄ may be C₁-C₄ alkoxy groups, and R₅ and R₆ may be hydrogen. Combinations of the above examples are also possible.

Flavolipids according to the present specification may also be produced semisynthetically. Semisynthetic production of flavolipids may isolate either flavolipids or flavolipid precursors from natural sources, which may be subsequently modified by chemical reactions. Such chemical reactions may include attachment of a variety of R₁ and/or R₂ groups according to formula (I). Semisynthetic production of flavolipids may provide an ecologically sustainable route to access to the full range of flavolipids that would be available through synthetic production. Semisynthesis of flavolipids may occur by enzymatic reaction or by chemical treatment of flavolipids or flavolipid precursors.

As with other types of surfactants, flavolipids of the above formula (I) may be incorporated into a variety of compositions. Such compositions include cosmetic compositions, such as those used to wash skin or hair; detergent compositions, such as those used to wash laundry or dishes; and surface cleansing compositions, such as glass cleaners, tile cleaners, or other surface cleansers familiar to an individual skilled in the art.

Distinct types of surfactants may provide benefits that other types of surfactants may not be able to provide; consequently, surfactants may be used in combination in order to provide the benefits of more than one class of surfactants to a single composition. In some cases, it is also possible to include a single type of surfactant.

In some cases, it may be advantageous to include a number of additional surfactants in a composition according to the present specification. Such additional surfactants may be selected from any number of surfactants, and include anionic surfactants, cationic surfactants, nonionic surfactants, zwitterionic surfactants and amphoteric surfactants. Each of these will be discussed, briefly, below. The provided examples of surfactants within each class are intended to be non-limiting, and any known surfactant may be suitable for use in a composition according to the present specification. The appropriate amount of each type of surfactant may vary greatly, as a function of the purpose of the composition; this will be discussed further below. The provided examples, or any component noted herein, is also intended to include salt forms, such as alkali or alkaline earth metal salts, and sulfate salts, citrate salts, methosulfate salts, chloride salts, bromide salts, and the like.

Anionic surfactants include surfactants that have an hydrophilic end which bears a negative charge at or near neutral pH. In some cases, anionic surfactants may bear a negative charge that may be neutralized in acidic solution. Examples of anionic surfactants include fatty acids, alkyl ether carboxylic acids, alkyl sulfates, alkyl ether sulfates, alkyl sulfonates, alkyl phosphates, alkyl ether phosphates, alkyl phosphonates, alkylbenzenesulfonates, and combinations thereof.

Cationic surfactants include surfactants that have a hydrophilic end which bears a positive charge at or near neutral pH. In some cases, cationic surfactants may bear a permanent positive charge, while in other cases, cationic surfactants may bear a positive charge that may be neutralized in basic solution. Examples of cationic surfactants include quaternary ammonium surfactants, tertiary amine surfactants, secondary amine surfactants, primary amine surfactants, esterquat surfactants, amidoamine surfactants, and combinations thereof.

Nonionic surfactants include surfactants that have a hydrophilic end that does not bear a charge at or near neutral pH. To achieve charge-neutrality while also providing a hydrophilic end, nonionic surfactants may, for example, employ hydroxyl groups in their hydrophilic end; hydroxyl groups may be deprotonated to bear an anionic charge in basic solution. Examples of nonionic surfactants include alkoxylated fatty alcohols, alkoxylated fatty esters, alkanolamides, alkyl glycosides, and combinations thereof.

Zwitterionic surfactants include surfactants that have a hydrophilic end that bears both a positive charge and a negative charge at or near neutral pH. In some cases, a zwitterionic surfactant may bear a permanent cationic charge and a pH-dependent anionic charge. In a further example, a zwitterionic surfactant may bear cationic and anionic charges that are both pH-dependent. Examples of zwitterionic surfactants include betaine surfactants, sultaine surfactants, surfactants bearing an secondary or tertiary amine and a carboxylate or sulfate group, and combinations thereof.

Amphoteric surfactants include surfactants that have a hydrophilic end that bears both an acidic hydrophilic group and a basic hydrophilic group. Amphoteric surfactants may behave in an acidic or basic manner, depending on the conditions. Amphoteric surfactants may include surfactants based on aliphatic amines having carboxy, sulfo or phosphono side chains. Amphoteric surfactants include such surfactants as N-alkyl glycines, N-alkyl propionic acids, N-alkyl aminobutyric acids, N-alkyl taurines, N-alkyl sarcosines and amine oxide surfactants.

For the purposes of the present specification, cosmetic compositions may be any surfactant composition that is applied to skin or hair, to achieve a cosmetic or cleansing effect. Non-limiting examples of such cosmetic compositions include toothpaste, shampoo, conditioner, body wash, rinse-off hand cleansers, leave-on hand cleansers, hair dyes, hair gel, hair mousse, hair spray, and combinations thereof.

A cosmetic composition according to the present specification, which may include a number of flavolipids according to formula (I), may have a number of advantages. Such advantages may include decreased skin irritation, decreased skin drying, decreased damage to hair fibers during hair treatment, improved cleaning efficacy, and may also provide desirable skin sensory attributes. Further advantages to the inclusion of flavolipids according to formula (I) in a cosmetic composition according to the present specification include an improved sustainability profile, both in regards to production and upon release into the environment.

A cosmetic composition according to the present specification may contain surfactants in an amount ranging from 0.001% to 60% by weight, relative to the total weight of the composition. The surfactants may be distributed in any appropriate manner. For example, a cosmetic composition according to the present specification may contain from 0.001% to 30% of a flavolipid according to formula (I), in combination with a number of surfactants selected from additional biosurfactants, anionic surfactants, cationic surfactants, nonionic surfactants, zwitterionic surfactants and amphoteric surfactants in an amount ranging from 0.001% to 30% by weight. In another example, a cosmetic composition according to the present specification may contain a flavolipid according to formula (I) in an amount ranging from 0.1% to 10% by weight, and may also contain a number of anionic surfactants in an amount ranging from 0.1% to 10% by weight, and may also contain a number of cationic surfactants in an amount ranging from 0.1% to 10% by weight, and may also contain a number of nonionic surfactants in an amount ranging from 0.1% to 10% by weight, and may also contain a number of zwitterionic or amphoteric surfactants in an amount ranging from 0.1% to 10% by weight. In a further example, a cosmetic composition according to the present specification may contain a number of flavolipids according to formula (I) in an amount ranging from 1% to 5% by weight, and may also contain a number of anionic surfactants in an amount ranging from 1% to 5% by weight, and may also contain a number of cationic surfactants in an amount ranging from 1% to 5% by weight, and may also contain a number of nonionic surfactants in an amount ranging from 1% to 5% by weight, and may also contain a number of zwitterionic or amphoteric surfactants in an amount ranging from 1% to 5% by weight, with all weights being relative to the total weight of the composition. While the above examples provide for the use of each type of surfactant, it is also possible for any example to omit a number of types of surfactants, instead using some-rather than all of the listed surfactant types within the noted ranges.

For the purposes of the present specification, detergent compositions may be any surfactant composition for textiles or surfaces, and is used in an aqueous solution to remove dirt, oil, or other debris from the material being cleansed. Detergent compositions may be rinsed away from the material being cleansed in a subsequent step. Non-limiting examples of detergent compositions include hand dishwashing detergent, dishwasher detergent, solid laundry detergent, liquid laundry detergent, liquid laundry pre-treatment, liquid fabric softener, and the like.

A detergent composition according to the present specification, which may include a number of flavolipids according to formula (I), may have a number of advantages. Such advantages include improved drying efficiency, improved removal of fats and oils, decreased spotting, decreased streaking, and improved removal of stains containing multivalent metal ions (for instance, iron, copper, manganese, and aluminum cations); such stains may include stains from food, berries, wine, blood, rust, chemicals, and antiperspirant. Further advantages to the inclusion of flavolipids according to formula (I) in a detergent composition according to the present specification include an improved sustainability profile, both in regards to production and upon release into the environment.

A detergent composition according to the present specification may contain surfactants in an amount ranging from 0.001% to 60% by weight, relative to the total weight of the composition. The surfactants may be distributed in any appropriate manner. For example, a detergent composition according to the present specification may contain from 0.001% to 30% of a flavolipid according to formula (I), in combination with a number of surfactants selected from additional biosurfactants, anionic surfactants, cationic surfactants, nonionic surfactants, zwitterionic surfactants and amphoteric surfactants in an amount ranging from 0.001% to 30% by weight. In another example, a detergent composition according to the present specification may contain a flavolipid according to formula (I) in an amount ranging from 0.1% to 10% by weight, and may also contain a number of anionic surfactants in an amount ranging from 5% to 20% by weight, and may also contain a number of cationic surfactants in an amount ranging from 0.1% to 10% by weight, and may also contain a number of nonionic surfactants in an amount ranging from 1% to 15% by weight, and may also contain a number of zwitterionic or amphoteric surfactants in an amount ranging from 0.1% to 10% by weight. In a further example, a detergent composition according to the present specification may contain a number of flavolipids according to formula (I) in an amount ranging from 1% to 5% by weight, and may also contain a number of anionic surfactants in an amount ranging from 5% to 15% by weight, and may also contain a number of cationic surfactants in an amount ranging from 1% to 5% by weight, and may also contain a number of nonionic surfactants in an amount ranging from 3% to 10% by weight, and may also contain a number of zwitterionic or amphoteric surfactants in an amount ranging from 1% to 5% by weight, with all weights being relative to the total weight of the composition. While the above examples provide for the use of each type of surfactant, it is also possible for any example to omit a number of types of surfactants, instead using some—rather than all—of the listed surfactant types within the noted ranges.

For the purposes of the present specification, surface cleansing compositions may be any surfactant composition which is applied to a surface to remove dirt, dust, oil, or other debris from the surface. Surface cleansing compositions may differ from detergent compositions in that surface cleansing compositions are wiped away, rather than rinsed away. Non-limiting examples of surface cleansing compositions include glass cleaners, tile cleaners, stone cleaners, general surface cleansing treatments, and combinations thereof.

A surface cleansing composition according to the present specification, which may include a number of flavolipids according to formula (I), may have a number of advantages. Such advantages include decreased skin irritation during cleansing with the composition, decreased stripes appearing on the surface following cleansing the surface, improved removal of dirt, improved removal of dust, and improved removal of oils and fats. Further advantages to the inclusion of flavolipids according to formula (I) in a surface cleansing composition according to the present specification include an improved sustainability profile, both in regards to production and upon release into the environment.

A surface cleansing composition according to the present specification may contain surfactants in an amount ranging from 0.001% to 60% by weight, relative to the total weight of the composition. The surfactants may be distributed in any appropriate manner. For example, a surface cleansing composition according to the present specification may contain from 0.001% to 30% of a flavolipid according to formula (I), in combination with a number of surfactants selected from additional biosurfactants, anionic surfactants, cationic surfactants, nonionic surfactants, zwitterionic surfactants and amphoteric surfactants in an amount ranging from 0.001% to 30% by weight. In another example, a surface cleansing composition according to the present specification may contain a flavolipid according to formula (I) in an amount ranging from 0.2% to 10% by weight, and may also contain a number of anionic surfactants in an amount ranging from 0.5% to 10% by weight, and may also contain a number of cationic surfactants in an amount ranging from 0.1% to 10% by weight, and may also contain a number of nonionic surfactants in an amount ranging from 0.5% to 10% by weight, and may also contain a number of zwitterionic or amphoteric surfactants in an amount ranging from 0.1% to 5% by weight. In a further example, a surface cleansing composition according to the present specification may contain a number of flavolipids according to formula (I) in an amount ranging from 0.5% to 5% by weight, and may also contain a number of anionic surfactants in an amount ranging from 0.5% to 5% by weight, and may also contain a number of cationic surfactants in an amount ranging from 0.5% to 5% by weight, and may also contain a number of nonionic surfactants in an amount ranging from 0.5% to 5% by weight, and may also contain a number of zwitterionic or amphoteric surfactants in an amount ranging from 0.5% to 5% by weight, with all weights being relative to the total weight of the composition. While the above examples provide for the use of each type of surfactant, it is also possible for any example to omit a number of types of surfactants, instead using some-rather than all of the listed surfactant types within the noted ranges.

Distinct types of surfactants may provide benefits that other types of surfactants may not be able to provide; consequently, surfactants may be used in combination in order to provide the benefits of more than one class of surfactants to a single composition.

In addition to surfactants, compositions according to the present specification may also include a number of additional ingredients. Examples of such additional ingredients include a carrier fluid, a conditioning agent, an emollient, a humectant, an exfoliant, a dye, a pigment, an oil, a wax, a silicone oil, a silicone wax, a vitamin, a vitamin derivative, a pH adjusting agent, an antibacterial agent, a UV stabilizer, a thickening agent, a chelating agent, an organic solvent, an opacifier, an enzyme, a corrosion inhibitor, a builder, a bleaching agent, a bleach activator, an optical brightener, an anticaking agent, a fragrance, and combinations thereof.

A carrier fluid may be a fluid that solubilizes or suspends the components of a composition according to the present specification, if such a composition is a liquid. Examples of suitable carrier fluids vary by application, but include water, lower alcohols, organic solvents, and combinations thereof. Lower alcohols may include a C₁-C₆ carbon chain that bears at least one alcohol group, including, for example, methanol, ethanol, ethylene glycol, n-propanol, isopropanol, 1,2-propylene glycol, 1,3-propylene glycol, n-butanol, isobutanol, tert-butanol, 1,2-butylene glycol, 1,3-butylene glycol, 1,4-butylene glycol, glycerol, and combinations thereof. Organic solvents may include carbon-based compounds that are liquid at standard ambient temperature and pressure (SATP, 298.15 Kelvin (K), 100 kiloPascals (kPa)). Examples of organic solvents include glycerol and glycols, such as 1,2-propylene glycol, ethylene glycol, polyethylene glycol, polypropylene glycol, as well as components that do not bear a hydroxyl group, such as acetone, acetonitrile, ethers and 1,4-dioxane. A carrier fluid may provide from 10% to 99% of a composition according to the present specification. For example, a carrier fluid may provide from 40% to 95% of a composition according to the present specification. In a further example, a carrier fluid may provide from 50% to 90%, such as from 70% to 85%, of a composition according to the present specification, with all amounts being percentages by weight, relative to the total weight of the composition.

A cosmetically suitable carrier may be a type of carrier fluid, which may be applied to skin or hair without producing an adverse reaction. Specific examples of cosmetically suitable carrier fluids include water, ethanol and isopropanol.

A conditioning agent may be a component which acts to preserve existing moisture by creating a hydrophobic barrier between air and moisturized skin or hair. Non-limiting examples of conditioning agents include the polyquaternium class of polymers, dimethicones, fatty alcohols, polyols, and certain cationic surfactants. A conditioning agent may be used in a cosmetic composition according to the present specification, and may be present in an amount ranging from 0.001% to 20% by weight. For example, a conditioning agent may be used in a cosmetic composition according to the present specification in an amount of from 0.01% to 10%, such as from 0.1% to 5% by weight, with all weights being relative to the total weight of the composition.

An emollient may be a component that acts to soften the outer layers of the skin. Emollients may prevent the evaporation of water in order to preserve hydration of the skin. Non-limiting examples of emollients include PEG-120 methyl glucoside dioleate, isopropyl laurate, isopropyl myristate, isopropyl palmitate, isodecyl myristate, isodecyl palmitate, methyl glucose laurate, polyglyceryl-6 tetraoleate, and the like. An emollient may be used in a cosmetic composition according to the present specification, and may be present in an amount ranging from 0.001% to 20% by weight. For example, an emollient may be used in a cosmetic composition according to the present specification in an amount of from 0.01% to 10%, such as from 0.1% to 5% by weight, with all weights being relative to the total weight of the composition.

A humectant may be a hygroscopic substance, or a substance that attracts water. Humectants may be added to a composition according to the present specification in order to maintain moisture on the skin or hair to which the composition is applied. Humectants may also be added to a composition according to the present specification in order to maintain moisture in the composition. Non-limiting examples of humectants that may be used in a composition according to the present specification include polyols, urea, panthenol, honey, aloe vera gels, glycerol, sorbitol, and glycols, such as propylene glycol and butylene glycol. Humectants may be present in a composition according to the present specification in an amount ranging from 0.01% to 20% by weight. For example, a humectant may be used in a composition according to the present specification in an amount ranging from 0.1% to 10% such as from 1% to 5% by weight, with all weights being relative to the total weight of the composition.

An exfoliant may be a particle that is a non-dissolvable solid. An exfoliant may be dispersed throughout an example of compositions according to the present specification, in order to act as an abrasive when the composition is used to cleanse the skin, hair, textiles, or solid surfaces. When used on skin, the abrasive properties of exfoliating particles may remove dead skin cells from the surface of the skin in order to allow deeper action of a surfactant, promote regeneration of the skin, and allow an emollient or humectant to access deeper layers of the skin. When used on textiles, the abrasive properties of exfoliating particles may increase the removal of stains or particulate matter from the textile by providing a scrubbing action. When used on solid surfaces, exfoliating particles may assist the removal of material that has accumulated on the solid surface. Non-limiting examples of exfoliating particles that may be used in a composition according to the present specification include actinidia chinensis (kiwi) seed, avena sativa (oat) bran, avena sativa (oat) kernel meal, carya illinoensis (pecan) shell powder, cocos nucifera (coconut) shell powder, egg shell powder, fragaria vesca (strawberry) seed, illite, silica, tin oxide, persea gratissima (avocado) fruit powder, and triticum vulgare (wheat) starch. An exfoliant may be present in a composition according to the present specification in an amount ranging from 0.01% to 15% by weight. For example, an exfoliant may be present in a composition according to the present specification in an amount ranging from 0.1% to 10%, such as from 0.25% to 5% by weight, with all weights being relative to the total weight of the composition.

Dyes and pigments may be compounds which confer color to a composition according to the present specification or a surface to which a composition according to the present specification is applied. Dyes and pigments may be added to a composition according to the present specification in order to imbue the composition with a consumer-acceptable color. Non-limiting examples of dyes and pigments which may be used in a composition according to the present specification include oxidation dyes, titanium dioxide, mica, iron oxides, ultramarines, violet 2, red 4, blue 1, blue 4, yellow 5, yellow 6, orange 4, and orange 5. Dyes and pigments may be present in a composition according to the present specification in an amount ranging from 0.0001% to 10% by weight. For example, dyes and pigments may be present in a composition according to the present specification in an amount ranging from 0.01% to 7% by weight, such as from 0.1% to 5% by weight, with all weights being relative to the total weight of the composition.

An oil may be a neutral, nonpolar substance that is a viscous liquid at standard ambient temperature and pressure (“SATP,” defined above). Oils may be included in an example of a composition according to the present specification in order to act as conditioning agents, viscosity modifiers, or to replenish natural oils on the skin. Non-limiting examples of oils that may be used in a composition according to the present specification include triglycerides, fatty alcohols, and mineral oils. An oil may be included in a composition according to the present specification in an amount ranging from 0.01% to 20%. For example, an oil may be included in a composition according to the present specification in an amount ranging from 0.1% to 10% by weight, such as from 0.5% to 5% by weight, with all weights being relative to the total weight of the composition.

A wax may be a compound that has a large fatty content, and is solid at SATP. Waxes may be malleable at SATP. Waxes may have a melting point at or above about 318 K. A wax may be included in a composition according to the present specification to improve rheological properties, modify the viscosity, act as conditioning agents, or provide other such properties to the composition. Non-limiting examples of types waxes that may be used in a composition according to the present specification include fatty esters, fatty ethers, hydrocarbons, primary alcohols, secondary alcohols, ketones and aldehydes; these agents include compounds such as isopropyl palmitate, candelilla wax, and beeswax. A wax may be included in a composition according to the present specification in an amount ranging from 0.01% to 15% by weight. For example, a wax may be included in a composition according to the present specification in an amount ranging from 0.1% to 10%, such as from 0.5% to 5% by weight, with all weights being relative to the total weight of the composition.

A silicone oil may be a type of oil that includes a siloxy backbone, which may correspond to a Si—(O—Si)ₙ chain. Silicone oils may have hydrocarbon groups attached to the siloxy backbone, or may also have further siloxy groups attached to the siloxy backbone; silicone oils may also have functional groups incorporated therein, including amines, alcohols, and ethers. Non-limiting examples of silicone oils that may be incorporated in compositions according to the present specification include dimethicone, cyclomethicone, dimethiconol, PEG-12 dimethicone, PEG-8 dimethicone, amodimethicone, alkyl methicones, and the like. Silicone oils may be included in a composition according to the present specification in amounts corresponding to the amounts provided above for oils.

A silicone wax may be a type of wax that includes a siloxy backbone, which may correspond to a Si—(O—Si)ₙ chain. Silicone waxes may have hydrocarbon groups attached to the siloxy backbone, or may have further siloxy groups attached to the siloxy backbone; silicone waxes may also have functional groups incorporated therein. Non-limiting examples of silicone waxes that may be used in compositions according to the present specification include alkyl methicones, Silwax A-08, Silwax C, Silwax D-02, Silwax F, Silwax S, and the like.

A vitamin may be an organic compound that an organism may require in limited quantities, and which the organism that may require the vitamin cannot synthesize from other precursors. Vitamins, or vitamin derivatives, may be included in an example of compositions according to the present specification as conditioning agents, preservatives, antioxidants, or to improve consumer acceptance of the composition. Non-limiting examples of vitamins that may be used in compositions according to the present specification include vitamin A (retinol), vitamin B₆ (pyroxidine), vitamin B₁₂ (cyancobalamin), vitamin C (ascorbic acid), vitamin E (tocopherols), and the like. For the purposes of the present specification, “vitamin” also includes vitamin derivatives and stereoisomers of vitamins, such as polyoxypropylene (2) polyoxyethylene (5) tocopherol ether, and isoascorbic acid. Vitamins may be included in a composition according to the present specification in amounts ranging from 0.0001% to 5% by weight. For example, a vitamin may be included in a composition according to the present specification in an amount ranging from 0.001% to 3%, such as from 0.01% to 1% by weight, with all weights being relative to the total weight of the composition.

A pH adjusting agent may be any agent which adjusts or controls the pH of the composition to the desired range. The desired pH range may depend on the end application of the composition; accordingly, pH adjusting agents include both acids and bases. Non-limiting examples of pH adjusting agents include acetic acid, citric acid, glycolic acid, lactic acid, tartaric acid, hydrochloric acid, sulfuric acid, ammonia, alkanolamines, alkali or alkaline earth metal hydroxides, alkali or alkaline earth metal carbonates, and the like. Agents to adjust the pH may be included in a composition according to the present specification in an amount ranging from 0.01% to 20% by weight. For example, a composition according to the present specification may include a pH adjusting agent in an amount ranging from 1% to 15%, such as from 3% to 10% by weight, with all weights being relative to the total weight of the composition.

A UV stabilizer may be a compound that absorbs ultraviolet light (light with a wavelength in the range of 280 nanometers to 400 nanometers). Exposure to ultraviolet light may be related to degradation of certain types of materials, and the inclusion of a UV stabilizer may slow or prevent the degradation of a composition according to the present specification. Non-limiting examples of UV stabilizers include hindered amine light stabilizers, avobenzone, octyl methoxycinnamate, ortho-hydroxy benzophenones, ortho-hydroxy phenyl triazines, and ortho-hydroxy phenyl hydrazines. UV stabilizers may be included in a composition according to the present specification in an amount ranging from 0.001% to 10% by weight. For example, a composition according to the present specification may include a UV stabilizer in an amount ranging from 0.01% to 5% by weight, such as from 0.1% to 2% by weight, with all weights being relative to the total weight of the composition.

An antibacterial agent may be an agent that assists in the removal of bacteria, kills bacteria, or arrests bacterial growth. Suitable non-limiting examples of antibacterial agents include antiseptics, benzethonium salts, benzalkonium salts, compounds that inhibit the 70S (bacterial) ribosome, compounds that reduce the integrity of the bacterial cell wall; such agents include ethanol, isopropanol, aminoglycosides, cephalosporins, lincosamides, tetracyclines, penicillins, and the like. A composition according to the present specification may include an antibacterial agent (other than ethanol or isopropanol) in an amount ranging from 0.01-5% by weight. For example, a composition according to the present specification may include an antibacterial agent in an amount ranging from 0.1% to 3% by weight, such as from 0.2% to 1% by weight, with all weights being relative to the total weight of the composition. If ethanol or isopropanol is used as a antibacterial agent, higher concentrations—such as at least 50% by weight, for example at least 60% by weight, may be used to achieve an antibacterial effect.

A thickening agent may be an agent that is included to increase the viscosity of a composition according to the present specification. Non-limiting examples of thickening agents include natural polymers, synthetic polymers, semi-synthetic polymers and inorganic thickeners; such agents include natural polymers such as guar gum, xanthan gum, collagen, silk, keratin, agar, carrageenan, polyquaternium polymers, carbomer, acrylates copolymers, zeolites and silicas. A thickening agent may be included in a composition according to the present specification in an amount ranging from 0.001% to 10% by weight. For example, a thickening agent may be included in a composition according to the present specification in an amount ranging from 0.01% to 5% by weight, such as from 0.1% to 1% by weight, with all weights being relative to the total weight of the composition.

A chelating agent may be a compound that coordinates metal ions. Certain chelating agents may also be able to act as preservatives, pH regulators, antibacterial agents, or provide other such properties to a composition according to the present specification. Non-limiting examples of chelating agents that may be used in a composition according to the present specification include natural polyacids, phosphate salts, bisphosphonates, aminocarboxylic acids, and combinations thereof. In some cases, flavolipids according to formula (I) may also be able to act as chelating agents. Chelating agents may be included in a composition according to the present specification at concentrations ranging from 0.001% to 5% by weight. For example, a composition according to the present specification may include a chelating agent in an amount ranging from 0.01% to 3% by weight, such as from 0.01% to 1% by weight, with all weights being relative to the total weight of the composition.

An opacifier may be a substance that causes a composition according to the present specification to be opaque. Non-limiting examples of opacifiers include titanium dioxide, cetyl alcohol, dolomite, rubber latex, silica, talc, and the like. A composition according to the present specification may include opacifiers in amounts ranging from 0.1% to 30% by weight. For example, a composition according to the present specification may contain opacifiers in an amount ranging from 1% to 20% by weight, such as from 3% to 10% by weight, with all weights being relative to the total weight of the composition.

An enzyme may be a biologically produced component that is able to decrease the activation energy of a chemical reaction; this decreasing of activation energy may be referred to as catalysis. For example, some enzymes may be hydrolysases, which may catalyze hydrolysis of a chemical bond. Such enzymes include proteases, lipases, amylases and cellulases. Enzymes may be substrate-specific, with varying degrees of specificity. For example, proteases may hydrolyze amide bonds in a protein backbone; certain proteases may be able to hydrolyze any proteinaceous amide, while other proteases may be specific for certain amino acid sequences; some proteases may also hydrolyze one or two amino acids from a terminus of a protein chain (exoproteases), while others may be able to hydrolyze a protein chain in the middle (endoproteases). Similar specificities may also exist for lipases (which catalyze the hydrolysis of fats) or cellulases (which catalyze the hydrolysis of polysaccharides). Enzymes may be included in small amounts in a composition according to the present specification, such as up to 1% by weight. For example, a composition according to the present specification may contain up to 0.5% by weight, such as up to 0.1% by weight, of an enzyme, with all weights being relative to the total weight of the agent. In a composition according to the present specification that includes enzymes, the enzymes may be provided such that the composition is stable; this may include encapsulation of the enzymes, and/or selection of the components of the composition to avoid undesirable degradation. Such degradation may result, for example, in a composition that employs a cellulose-based thickening agent in combination with a cellulase enzyme.

A corrosion inhibitor may be a compound that prevents the corrosion of a material that comes in contact with a composition according to the present specification. Corrosion inhibitors may act to form a coating on a surface that may prevent access to the surface of a corrosive material in a composition according to the present specification. Non-limiting examples of corrosion inhibitors include cyclohexylamines, diammonium phosphates, nitromethane, benzotriazoles, potassium silicate, sodium aluminate, sodium nitrite, triisopropanolamine, and the like. A detergent or surface cleansing composition according to the present specification may contain a corrosion inhibitor in an amount ranging from 0.01% to 10% by weight. For example, a detergent or surface cleansing composition according to the present specification may contain a corrosion inhibitor in an amount ranging from 0.1% to 5% by weight, such as from 0.5% to 3% by weight, with all weights being relative to the total weight of the composition.

A builder may be a compound which complexes with ions that contribute to water hardness, such as calcium and magnesium ions. Non-limiting examples of builders include, for example, carbonate salts, sulfate salts, silicates, zeolites, polymers, organic acids, and the like. Builders that are acidic may be added in either their acid form or their salt form. Non-limiting examples of polymers which may be able to act as builders include polyaspartic acid, polycarboxylic acids such as polyacrylic acid, polymethacrylic acid, and acrylic acid copolymers. A detergent or surface cleansing composition according to the present specification may include builders in an amount ranging from 0.001% to 50% by weight. For example, a liquid detergent or surface cleansing composition according to the present specification may include builders in an amount ranging from 0.01% to 4% by weight, such as from 0.15% to 1.5% by weight, with all weights being relative to the total weight of the composition. In another example, a solid or particulate detergent or surface cleansing composition according to the present specification may include builders in an amount ranging from 15% to 40% by weight, such as from 20% to 30% by weight, with all weights being relative to the total weight of the composition. In a further example, a builder may be included in a solid state composition according to the present specification and may act as a solid carrier.

A bleaching agent may be an oxidative agent that removes or modifies colors. Some bleaching agents may act by oxidizing carbon-carbon double bonds in conjugated systems such that the resultant compound is white or colorless. Non-limiting examples of bleaching agents include percarbonates, peroxides, persulfates, peracids and perborates. For example, hydrogen peroxide may be suitable for use as a bleaching agent in some examples of a composition according to the present specification. The amount of bleaching agent that may be incorporated into a composition according to the present specification may vary as a function of the intended application. For example, certain types of cosmetic compositions according to the present specification may not contain any bleaching agent, while other types of cosmetic compositions—such as hair bleaching compositions, skin lighteners, oxidative compositions as part of a hair dyeing kit, et cetera—may contain from 1% to 20% by weight of a bleaching agent. In another example, a cosmetic composition according to the present specification may contain a bleaching agent in an amount ranging from 3% to 15% by weight, such as from 4% to 10% by weight, with all weights being relative to the total weight of the composition. A detergent or surface cleansing composition according to the present specification may contain from 1% to 30% by weight of a bleaching agent. For example, a detergent or surface cleansing composition according to the present specification may contain from 1% to 10% by weight, such as from 2% to 5% by weight of a bleaching agent. In another example, a detergent or surface cleansing composition according to the present specification may contain from 5% to 25% by weight of a bleaching agent, such as from 15% to 20% by weight, with all weights being relative to the total weight of the composition.

A bleach activator may be a component which assists the effect of a bleaching agent. Bleach activators may be compounds that contain a number of N- or O-acyl groups, such as anhydrides, esters, imides, and acylated imidazoles or oximes. Specific non-limiting examples of bleaching activators include tetra-acetylethylenediamine (TAED), manganese-based bleach activators, pentacetylglucose (PAG), and the like. Bleach activators may be included in a detergent or surface-cleansing composition according to the present specification in an amount ranging from 0.01% to 20% by weight. For example, a liquid detergent or surface cleansing composition according to the present specification may contain a bleach activator in an amount ranging from 0.1% to 5% by weight, such as from 1% to 4% by weight, with all weights being relative to the total weight of the composition. In another example, a solid or particulate detergent or surface cleansing composition according to the present specification may contain a bleach activator in an amount ranging from 5% to 20% by weight, such as from 8% to 15% by weight, with all weights being relative to the total weight of the composition.

An optical brightener may be an agent that is added to a detergent or surface cleansing composition to enhance the appearance of the composition or surfaces/textiles to which the composition is applied. Some optical brightening agents may absorb light in the ultraviolet and violet region (a wavelength of 300 to 420 nanometers), and re-emit light in the blue region (a wavelength of 420 to 470 nanometers). Some times of optical brighteners may also be called fluorescent whitening agents. Non-limiting examples of optical brightening agents include stilbenes, diphenylstyryl compounds, and compounds bearing a diethanolamino group, a methylamino group, an aniline group, or a 2-methoxyethylamino group. Optical brighteners may be included in a detergent or surface cleansing composition according to the present specification in an amount ranging from 0.01% to 5% by weight. For example, a detergent or surface cleansing composition according to the present specification may include optical brighteners in an amount ranging from 0.05% to 3% by weight, such as from 0.1% to 0.5% by weight, with all weights being relative to the total weight of the composition.

An anti-caking agent may be an agent that prevents or reduces agglomeration of a composition according to the present specification that is provided in a solid or particulate form. Non-limiting examples of anti-caking agents that may be used in a composition according to the present specification include amorphous silicate material, aluminosilicates, sodium bicarbonate, talcum powder, and tricalcium phosphate. A composition according to the present specification that is provided in a solid or particulate form may include an anti-caking agent in an amount ranging from 0.01% to 40% by weight. For example, a composition according to the present specification that is provided in a solid or particulate form may include an anti-caking agent in an amount ranging from 0.1% to 35% by weight, such as from 1% to 2% by weight, with all weights being relative to the total weight of the composition.

A fragrance may be a component or composition that produces an olfactory sensation in an individual. A fragrance may contain a single component, or may also be a mixture of multiple separate components. Non-limiting examples of fragrance components that may be used in a composition according to the present specification include aldehydes, ketones, aromatic hydrocarbons, aromatic alcohols, and combinations thereof. Examples of fragrance ingredients may include alpha-hexyl cinnamal, vanillin, citral, eugenol, geraniol, limonene, and citronellol. A composition according to the present specification may include a fragrance in an amount ranging from 0.001% to 5% by weight. For example, a composition according to the present specification may include a fragrance in an amount ranging from 0.01% to 2% by weight, such as from 0.1% to 1% by weight, with all weights being relative to the total weight of the composition.

A composition according to the present specification may also contain further additive ingredients in appropriate quantities, depending on the end application. Examples of such further ingredients include peroxide stabilizers, reducing agents, encapsulation materials, antiredeposition agents, and the like.

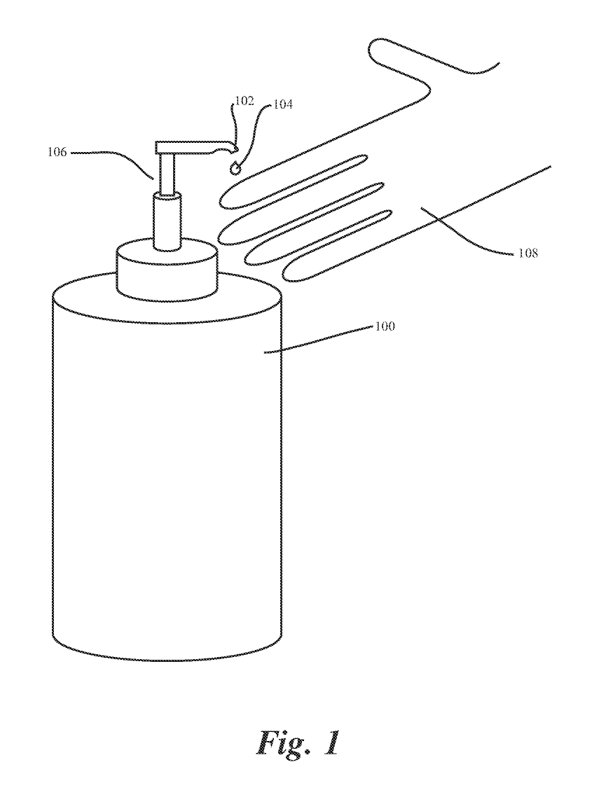

A composition according to the present specification may be provided in any suitable container, which may also be equipped with a dispensing apparatus. Examples of suitable containers are described in FIGS. 1, 2 and 3; the examples shown in FIGS. 1, 2 and 3 are not intended to be limiting. Any type, shape or size of container may be suitable for use with a composition according to the present specification. Additionally, a container that houses a composition according to the present specification may be prepared from any suitable material, such as clear or transparent plastic, opaque plastic, metal, cardboard, and the like.

Turning now to the figures, FIG. 1 shows diagram of an exemplary container dispensing a cosmetic composition that includes a flavolipid, according to an example of the principles described herein. A cosmetic composition (104) that includes a flavolipid may be housed within a container (100). Such a container (100) may include a pump-action dispenser (106), which may include an aperture (102) through which the cosmetic composition (104) that includes a flavolipid is dispensed. A user may dispense the cosmetic composition (104) that includes a flavolipid onto a hand (108). While the diagram shown in FIG. 1 may provide a pump-action dispenser (106), other types of dispensers (106) may also be used with a composition (104) according to the present specification. For example, the dispenser (106) could be aerosol-based, a simple cap to pour out the composition (104), or a foaming dispenser (106). Other types of dispensers (106) may also be suitable. A user may also dispense a cosmetic composition (104) directly onto the site of intended use, which may or may not be a hand (108). For example, a user may dispense a cosmetic composition (104) that includes a flavolipid directly onto hair. A container (100) according to the present specification may be any shape or size, and may be prepared from any appropriate materials.

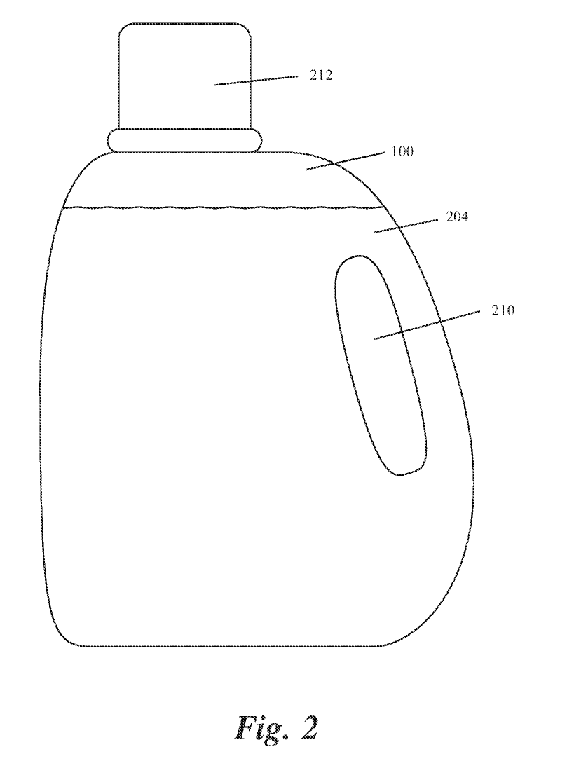

FIG. 2 shows a diagram of an exemplary container holding a detergent composition that includes a flavolipid, according to an example of the principles described herein. In the diagram of FIG. 2, the detergent composition (204) is housed within a container (100). The container (100) may be equipped with a handle (210), and may also be equipped with a lid assembly (212). The detergent composition (204) may be provided in any suitable form, for example as a liquid detergent composition (204) or a solid detergent composition (204). A solid detergent composition (204) may be provided as a powder, granulated particles, extruded particles, flakes, chunks, or in any suitable presentation of a solid. The handle (210) shown in FIG. 2 is exemplary; such a handle (210) on a container (100) may be of any suitable size and shape. The container (100) may be made from any suitable material, and may be provided in any appropriate configuration. For example, the container (100) could be a bottle, tub, box, or the like. Certain types of containers (100) may benefit from a lid (212), such as the one shown in FIG. 2. Such a lid (212) may, in some examples, also be used to measure the detergent composition (204) prior to use.

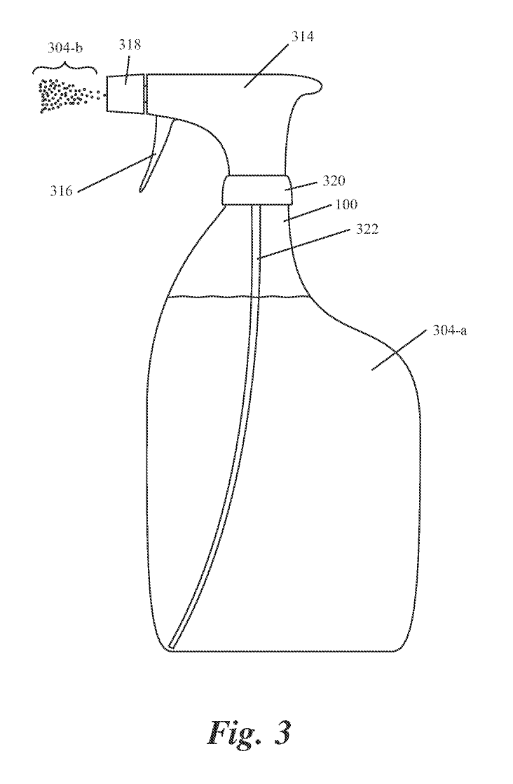

FIG. 3 shows a diagram of an exemplary container dispensing a surface cleansing composition that includes a flavolipid, according to an example of the principles described herein. In the diagram of FIG. 3, the surface cleansing composition (304) that includes a flavolipid is housed within a spray bottle, and is shown prior to dispensing (304-a) and immediately following dispensing (304-b); the spray assembly (314) may be provided such that the surface cleansing composition prior to dispensing (304-a) is identical to the surface cleansing composition immediately following dispensing (304-b). In another example, the spray assembly (314) may be provided to alter the surface cleansing composition (304) during the dispensing process; for example, the spray assembly (314) may include a foaming apparatus, such that the surface cleansing composition (304-a) is mixed with a gas, which may be air, to provide the surface cleansing composition immediately following dispensing (304-b). A spray assembly (314) may include a spray handle (316), a dispensing nozzle (318), and an attachment element (320). A spray handle (316) may provide an actuation lever that allows the surface cleansing composition (304) that includes a flavolipid to be dispensed (304-b). A dispensing nozzle (318) may provide an aperture through which the surface cleansing composition (304) that includes a flavolipid is dispensed. An attachment element (320) may provide a connection between a spray assembly (314) and a container (100), which may house the surface cleansing composition (304-a) that includes a flavolipid prior to dispensing. A spray assembly (314) may also include a dip tube (322), which may draw the surface cleansing composition (304-a) into a spray assembly (314) to be dispensed.

As noted above, a container (100) may be of any suitable size and shape, and may be made from any suitable material. The above descriptions of the types of containers (100) for each type of composition (104, 204, 304) may also be used for the other types of compositions (104, 204, 304) according to the present specification. For example, a cosmetic composition (104) may also be provided in a spray bottle, which may include a spray assembly (314). Similarly, a surface cleansing composition (304) that includes a flavolipid may also be provided in a container (100) that includes a lid (212).

The components of each type of container (100) shown in FIGS. 1, 2 and 3 may be arranged in any suitable manner. For example, a dip tube (322) on a spray assembly (314) may be provided to also include a pre-filter. In another example, a dip tube (322) on a spray assembly (314) may be integrated into the structure of the container (100), so as to maximize the quantity of the composition (104, 204, 304) that may be dispensed from the container (100).

A spray assembly (314) may be equipped with a rotating dispensing nozzle (318). A rotating dispensing nozzle (318) may be configured to have an “on” orientation, in which a composition (304) may be dispensed, and may also have an “off” orientation, in which a composition (304) may not be dispensed.

### EXAMPLES

The examples that follow indicate cosmetic, detergent, and surface cleansing compositions that may be prepared in accordance with the present specification. Unless otherwise indicated, the stated quantities are percentages by weight.

Each example below may be prepared according to a variety of methods.

### Example 1

### Rinse-Off Liquid Hand Cleanser

TABLE (I)

| Formulation Example 1 | Wt. % |
|---|---|
| Flavolipid according to | 2-6 |
| formula (I) | |
| Anionic Surfactant(s) | 1-6 |
| Nonionic Surfactant(s) | 0-5 |
| Zwitterionic Surfactant(s) | 0-10 |
| Polymeric Thickener | 0.1-5 |
| Preservative(s) | 0.2-2 |
| Exfoliant(s) | 0-5 |
| Water | q.s. 100 |

### Example 2

### Leave-on Liquid Hand Cleanser

TABLE (II)

| Formulation Example 1 | Wt. % |
|---|---|
| Flavolipid according to | 0.1-5 |
| formula (I) | |
| Benzalkonium Chloride | 0-5 |
| Conditioning Agent(s) | 0-5 |
| Silica | 1-6 |
| Water | q.s. 100 |

### Examples 3-10

### Liquid Laundry Detergent

TABLE (III)

| | Formulation Example | | | | | | | |
|---|---|---|---|---|---|---|---|---|
| | 3 | 4 | 5 | 6 | 7 | 8 | 9 | 10 |
| Flavolipid according to formula (I) | 2% | 2% | 2% | 2% | 2% | 2% | 2% | 2% |
| Sodium C10-13 Alkyl | 9% | 10% | 6% | 7% | 5% | 15% | 15% | 9% |
| Benzenesulfonate | | | | | | | | |
| C12-18 Pareth-7 | 8% | 9% | 6% | 7% | 5% | 6% | 11% | 10% |
| C12-14 Alkyl Ether Sulfate with | — | — | 8% | 7% | 10% | 2% | 2% | 5% |
| 2EO | | | | | | | | |
| C12-18 Fatty Acids, Sodium Salt | 4% | 3% | 3% | 3% | 4% | 2% | 4% | 7% |
| Citric Acid | 2% | 3% | 3% | 2% | 2% | 2% | 2% | 3% |
| Sodium Hydroxide, 50% | 3% | 3% | 2% | 3% | 3% | 3% | 3% | 4% |
| Boronic Acid | 1% | 1% | 1% | 1% | 1% | 1% | 1% | 1% |
| Enzyme (Amylase, Protease, | + | + | + | + | + | + | + | + |
| Cellulase) | | | | | | | | |
| Fragrance | 1% | 0.5% | 0.5% | 1% | 1% | 1% | 1% | 1% |
| Propanediol | — | — | — | — | — | 5% | 5% | — |
| Ethanol | 1.5% | 1.5% | 1.5% | 1.5% | 1.5% | 1.5% | 1.5% | 5% |
| Polyvinyl Alcohol/Maleic Acid | 0.1% | — | 0.1% | — | — | — | — | — |
| Copolymer | | | | | | | | |
| Optical Brightening Agents | — | 0.1% | — | 0.1% | 0.2% | 0.2% | 0.2% | 0.2% |
| Opacifiers | 0.2% | — | — | — | — | — | — | — |
| Sodium Phosphate | 0.5% | 0.5% | 0.5% | 0.5% | 0.5% | 0.5% | 0.5% | 0.5% |

| | |
|---|---|
| Water | q.s. 100 |

### Example 11

### Particulate Laundry Detergent

| Formulation Example 11 | Wt. % |
|---|---|
| Flavolipid according to formula (I) | 5-10 |
| Anionic surfactant(s) | 5-20 |
| Nonionic surfactant(s) | 3-10 |
| Builder(s) | 20-40 |
| pH Adjusting Agent(s) | 2-8 |
| Bleaching Agent(s) | 18-24 |
| Bleach Activator(s) | 8-14 |
| Antiredeposition Agent(s) | 0-3 |
| Optical Brightener(s) | 0-2 |
| Anti-caking Agent(s) | 0-2 |
| Fragrance | 0-2 |
| Enzymes (coarse grade) | 0.5-4.5 |
| Water | 0.5-6 |

### Example 12

### Machine Dishwashing Detergent

TABLE (V)

| Formulation Example 12 | Wt. % |
|---|---|
| Flavolipid according to formula (I) | 0-10 |
| Sodium Citrate | 15-20 |
| Etidronic Acid | 2.5-7.5 |
| Methyl Glycine Diacetic Acid | 0-25 |
| Sodium Disilicate | 5-35 |
| Sodium Carbonate | 12.5-25 |
| Sodium Percarbonate | 10-15 |
| Manganese-based Bleach Activator | 0.02-0.5 |
| Tetraacetylethylenediamine | 2-3 |
| Ethoxylated Nonionic Surfactant | 2.5-10 |
| (20-40 EO) | |
| Polycarboxylic Acid | 5-10 |
| Cationic Polymer | 0.25-0.75 |
| Crosslinked Polyvinylpyrrolidone | 0-1.5 |
| Protease | 1.5-5 |
| Amylase | 0.5-3 |
| Benzotriazole | 0-0.5 |
| Fragrance | 0.05-0.15 |
| Dye(s) | 0-1 |
| Zinc Acetate | 0.1-0.3 |
| Sodium Sulfate | 0-25 |
| Water | 0-1.5 |
| Citric Acid (pH adjusting agent) | 1-1.5 |
| Processing Aids | 0-5 |

### Example 13

### Hand Dishwashing Detergent

TABLE (VI)

| Formulation Example 13 | Wt. % |
|---|---|
| Flavolipid according to formula (I) | 0-10 |
| Anionic Surfactant(s) | 5-25 |
| Nonionic Surfactant(s) | 1-15 |
| Amphoteric Surfactant(s) | 1-20 |
| Zwitterionic Surfactant(s) | 0-15 |
| Alkali Metal Halide(s) | 0-5 |
| Thickening Agent(s) | 0-5 |
| Cationic Polymer(s) | 0-5 |
| Preservative(s) | 0-1 |
| Water/Ethanol Mixture (70:30) | q.s. 100 |

### Example 14

### Glass Cleaner

TABLE (VII)

| Formulation Example 14 | Wt. % |
|---|---|
| Flavolipid according to formula (I) | 0-5 |
| Ammonium Hydroxide (pH Adjuster) | 0.1-1 |
| Ethanol | 5-10 |
| Fragrance | 0-0.2 |
| Water | q.s. 100 |

### Example 15

### Stone Cleaner

| Formulation Example 15 | Wt. % |
|---|---|
| Flavolipid according to formula (I) | 0-5 |
| Monoethanolamine (pH Adjuster) | 0.1-1 |
| Nonionic Surfactant(s) | 0-5 |
| Anionic Surfactant(s) | 0-5 |
| Amphoteric Surfactant(s) | 0-5 |
| Fragrance | 0-0.5 |
| Water | q.s. 100 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A surfactant composition, comprising:
a number of flavolipids according to formula (I), or salts thereof, 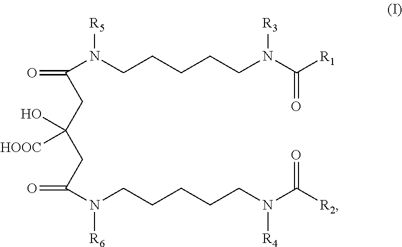
in which:
R₁ and R₂, independently of one another, are selected from:
linear or branched C₄-C₂₀ alkyl radicals;
linear or branched C₄-C₂₀ alkenyl radicals; and
linear or branched C₄-C₂₀ hydroxyalkyl radicals; and
R₃, R₄, R₅ and R₆, independently of one another, are selected from hydrogen, hydroxyl, C₁-C₄ alkyl radicals, C₁-C₄ alkenyl radicals, C₁-C₄ alkoxy radicals, and C₁-C₄ hydroxyalkyl radicals; and
a number of additional surfactants.

2. The surfactant composition of claim 1, in which the number of flavolipids of formula (I) are present in an amount ranging from 0.001% to 30% by weight, relative to the total weight of the composition.

3. The surfactant composition of claim 1, in which the number of additional surfactants are present in an amount ranging from 0.001% to 30% by weight, relative to the total weight of the composition.

4. The surfactant composition of claim 1, further comprising at least one of a conditioning agent, an emollient, a humectant, an exfoliant, a dye, a pigment, an oil, a wax, a silicone oil, a silicone wax, a vitamin, a vitamin derivative, a pH adjusting agent, an antibacterial agent, and a thickening agent, a chelating agent, an opacifier, a UV stabilizer, a corrosion inhibitor, a builder, a bleaching agent, a bleach activator, a pH adjusting agent, an optical brightener, a dye, an anti-caking agent, water, and an organic solvent.

5. The surfactant composition of claim 1, in which the number of additional surfactants comprises an anionic, nonionic or cationic surfactant.

6. The surfactant composition of claim 1, further comprising a carrier selected from a carrier fluid and a builder.

7. The surfactant composition of claim 1, in which the composition is a cosmetic composition for skin or hair.

8. The surfactant composition of claim 1, in which the composition is a detergent composition for textiles.

9. The surfactant composition of claim 8, further comprising an enzyme selected from a protease, an amylase, a cellulase and combinations thereof.

10. The surfactant composition of claim 1, in which the composition is a surface cleansing composition.

11. The surfactant composition of claim 10, further comprising an enzyme selected from a protease and an amylyase.

12. The surfactant composition of claim 10, in which the composition is a dishwashing detergent.

13. The surfactant composition of claim 1, in which R₃ and R₄ are hydroxyl, and R₅ and R₆ are hydrogen.

14. The surfactant composition of claim 1, in which R₁ and R₂ are independently selected from branched C₄-C₂₀ alkenyl radicals bearing a single carbon-carbon double bond.

15. The surfactant composition of claim 1, in which the number of additional surfactants comprises at least two surfactants, each selected, independently of one another, from anionic, nonionic, cationic, amphoteric and zwitterionic surfactants.

16. A cleansing composition for cleansing textiles or surfaces, comprising:
a number of flavolipids according to formula (I), or salts thereof, 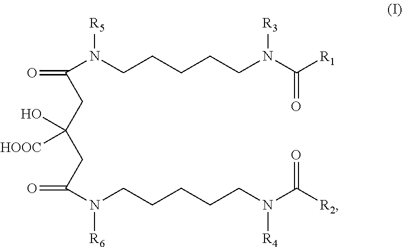
in which:
R₁ and R₂, independently of one another, are selected from:
linear or branched C₄-C₂₀ alkyl radicals;
linear or branched C₄-C₂₀ alkenyl radicals; and
linear or branched C₄-C₂₀ hydroxyalkyl radicals; and
R₃, R₄, R₅ and R₆, independently of one another, are selected from hydrogen, hydroxyl, C₁-C₄ alkyl radicals, C₁-C₄ alkenyl radicals, C₁-C₄ alkoxy radicals, and C₁-C₄ hydroxyalkyl radicals; and
a liquid carrier, comprising at least two miscible carrier fluids.

17. The cleansing composition of claim 16, in which R₃ and R₄ are hydroxyl and R₅ and R₆ are hydrogen, and R₁ and R₂ are, independently of one another, selected from C₄-C₂₀ alkenyl radicals bearing a single carbon-carbon double bond.

18. The cleansing composition of claim 16, further comprising a number of additional surfactants selected from anionic, cationic, nonionic, zwitterionic and amphoteric surfactants.

19. The cleansing composition of claim 16, in which the number of flavolipids are present in an amount ranging from 0.001% to 30% by weight, relative to the total weight of the cleansing composition.

20. A consumer product for use as a laundry detergent, comprising:
a number of flavolipids according to formula (I), or salts thereof, 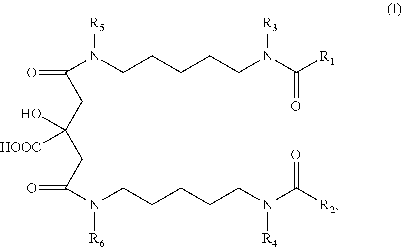
in which:
R₁ and R₂, independently of one another, are selected from:
linear or branched C₄-C₂₀ alkyl radicals;
linear or branched C₄-C₂₀ alkenyl radicals; and
linear or branched C₄-C₂₀ hydroxyalkyl radicals; and
R₃, R₄, R₅ and R₆, independently of one another, are selected from hydrogen and hydroxyl;
a number of anionic surfactants;
a number of nonionic surfactants;
a number of enzymes selected from proteases, amylases and cellulases;
a number of builders; and
a liquid carrier.
